(19) United States
(12) Patent Application Publication
(10) Pub. No.: US 2016/0157834 A1
(43) Pub. Date: Jun. 9, 2016

(54) URINE SPECIMEN COLLECTION DEVICE

(71) Applicant: Thomas Goldsmith, Huntington, NY (US)

(72) Inventor: Thomas Goldsmith, Huntington, NY (US)

(21) Appl. No.: 14/564,748

(22) Filed: Dec. 9, 2014

(51) Int. Cl. A61B 10/00

(52) U.S. Cl. CPC A61B 10/007

(57) ABSTRACT

A container for collecting a urine specimen has an outer wall, an inner shell and a lid structured to be removeably attached to an upper rim of the container for covering an open top. The inner shell extends down from the upper rim in spaced relation to the outer wall and terminates above a bottom of the container to form a pocket between the inner shell and the outer wall. The pocket traps and captures a suitable testing volume of the specimen within the container in the event the lid is not adequately secured to the container and a portion of the specimen leaks from the container during transport to a testing facility.

### BACKGROUND OF THE INVENTION

1. Field of the Invention

The present invention relates to urine specimen collection devices and, more particularly, to a urine specimen collection device adapted for capturing a portion of the urine specimen in the event of a spill.

2. Discussion of the Related Art

Urine collection devices are provided for the collection of urine for subsequent analysis. While there exists a variety of urine collection devices ranging from simple plastic cups to intricate collection devices intended to collect specific volumes of urine at various points in the micturition process, the most common urine collection devices include a plastic container with a snap-fit plastic lid. Typically, these plastic containers are filled with urine by a patient and the lid is subsequently snapped on. The filled container is then transported (generally with many other filled containers) to a testing facility (i.e., laboratory). During the transportation process, the containers can tip over and, if the snap-fit lid was not properly secured to the container, can result in spillage of the urine sample wherein the contents entirely escape the container. Testing facilities are not permitted to draw urine samples from any location other than directly from the container; therefore, any urine contents that exit the container are considered contaminated and cannot be tested. This results in lost revenues to the testing facility.

Accordingly, there exists a need for a urine specimen collection device adapted for capturing or otherwise trapping a portion of a urine sample in the event that the collection container cap is not properly secured during transportation of the sample. Moreover, there exists a need for such a urine specimen collection device that can be readily adapted to a standard urine collection container, thereby allowing for ease of production without a significant increase in manufacturing costs. Alternatively, the entire structure of the specimen collection container of the present invention can be manufactured as one piece (excluding the lid) from a single mold.

### OBJECTS AND ADVANTAGES OF THE INVENTION

It is an object of the present invention to provide a urine specimen collection device adapted for capturing and preventing loss of at least a portion of a urine sample contained therein in the event that the urine container cap is not properly secured to the container.

It is another object of the present invention to provide a urine specimen collection device that is inexpensive to manufacture and easily adaptable to existing urine collection containers.

It is another object of the present invention to provide a urine specimen collection device that permits pouring of a urine sample via the open end of the container where a standard volume of the urine sample exists.

It is another object of the present invention to provide a urine specimen collection device wherein the center of the container is not obstructed and permits use of a pipette to extract a urine sample therefrom.

It is yet another object of the present invention to provide a urine specimen collection device that may be produced by easily adapting manufacturing techniques used to produce currently existing urine collection containers known in the art.

It is still a further object of the present invention to provide a urine specimen collection device that can be manufactured as a one piece unitary structure from a single mold if desired.

It is still a further object of the present invention to provide a urine specimen collection device that can accommodate a point of care chemical reagent test indicator for rapid detection of the presence of one or more substances in the collected urine.

These and other objects and advantages will be readily apparent with reference to the following description and accompanying drawings.

### SUMMARY OF THE INVENTION

The present invention is directed to a urine sample collection device including an open-ended container, an inner shell fixable to an upper rim surrounding the opening of the container, and a lid configured to releasably attach to the upper rim for covering the opening of the container when secured thereto. The lid can be of the “screw-on” type that engages screw threads on the side of the rim of the container or, alternatively, the container and lid can be made to allow the lid to snap on to the upper rim of the container. The inner shell includes an inner channel extending between opposing open ends and is sized and configured to extend from the upper rim into the cavity of the container. The inner shell is tapered or otherwise curved in relation to the container wall such that a pocket is formed between the inner shell and the container wall at the upper rim of the container where the inner shell and container are fixed together. In the event that the lid of the urine specimen collection device is not adequately secured to the container when in transport to a testing facility, at least a portion of the specimen will become trapped in the pocket and not exit the container. The pocket surrounding the inner shell is sized and configured to capture a volume of the specimen suitable for testing.

The urine sample collection device can be design to be readily adapted to existing urine specimen collection containers wherein the inner shell is received within the existing container and bonded or otherwise fixed to the upper rim of the container. This may significantly reduce manufacturing costs by allowing manufacturers of existing urine collection containers to simply add the inner shell to their existing containers. Alternatively, the urine sample collection device of the present invention can be made as a one piece unitary structure (excluding the lid) from a single mold, or the entire device may be newly molded as two or more pieces and then bonded or otherwise fixed together as one piece.

In a further embodiment of the present invention, the urine sample collection container can be made to accommodate a point of care chemical reagent test indicator for onsite detection and indication of the presence of one or more substances in the urine within minutes of collection. For example, a point of care testing and indicator device can be fitted within the pocket between the inner shell and the wall of the outer container of the present invention during the manufacturing process.

### BRIEF DESCRIPTION OF THE DRAWINGS

For a fuller understanding of the nature of the present invention, reference should be made to the following detailed description taken in conjunction with the accompanying drawings in which:

FIG. 1 is a side elevational view, shown in cross-section, of the urine sample collection device of the present invention according to one embodiment, illustrating the container and an inner shell fixed thereto;

FIG. 2 is a side elevational view, shown in cross-section, of the urine sample collection device of the present invention according to another embodiment.

FIG. 3 is a side elevational view, shown in cross-section, of the urine sample collection device of the present invention according to one embodiment, illustrating a container on its side with a lid improperly attached thereto and permitting escape of a portion of the urine sample while capturing a portion of the sample in the pocket; and

FIG. 4 is a side elevational view, shown in cross-section, illustrating the inner shell being inserted into the container during the manufacturing process for attachment thereto.

Like reference numerals refer to like parts throughout the several views of the drawings.

### DETAILED DESCRIPTION OF THE PREFERRED EMBODIMENT

Referring to the several views of the drawings, the urine sample collection device of the present invention is shown and is generally indicated as 10.

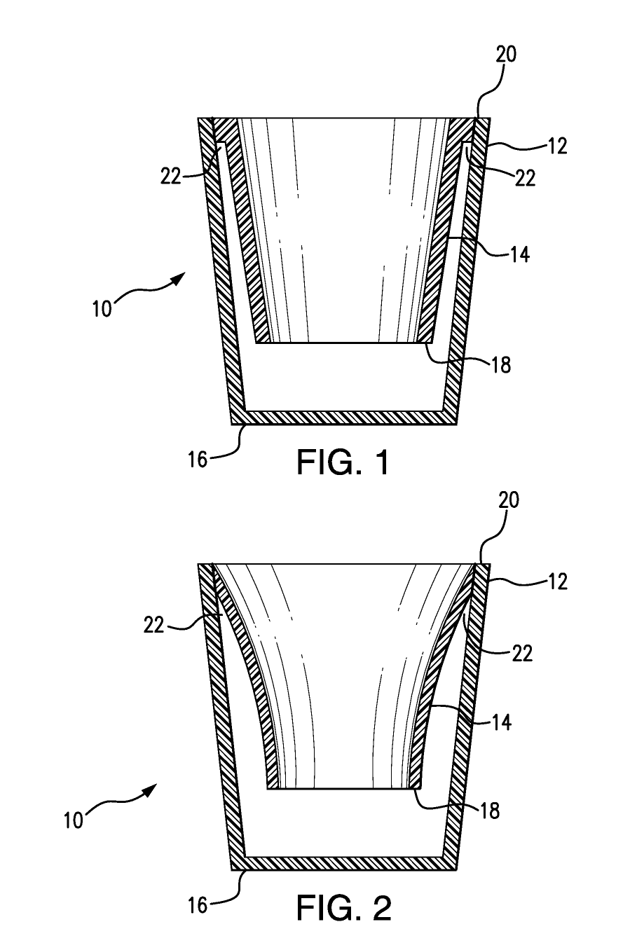

Referring initially to FIG. 1, the urine sample collection device 10 includes an open-ended container 12 and an inner shell 14 fixable thereto. The container 12 includes a sealed bottom end 16 that is set apart from the open bottom end 18 of the inner shell 14, which is fixed to an upper rim 20 surrounding the opening of the container 12. The inner shell 14 surrounds an inner channel extending between opposing open ends and is sized and configured to extend from the upper rim 20 into the cavity of the container 12. In one embodiment, as shown in FIG. 1, the inner shell 14 is tapered in relation to the wall of the container 12 such that a pocket 22 is formed between the inner shell 14 and the container 12 at the upper rim 20.

Referring to FIG. 2, an alternative embodiment of the inner shell 14 includes a convexly curved surface such that a pocket 22 is formed between the inner shell 14 and the walls of the container 12 at the upper rim 20.

A lid 24, such as a snap-fit lid, is configured to be releasably attached to the upper rim 20 for covering the opening of the container 12 and preventing spillage of contents therein. Use of snap-fit lids for securing urine samples in a container during transportation of the samples to a testing facility is well known in the art. Alternatively, the lid 24 can be of the “screw-on” type which is also well known in the art. In this instance, the lid 24 and upper rim 20 of the container would be provided with correspondingly engaging screw-threads to allow the lid 24 to be easily secured and removed from the open top of the container 12.

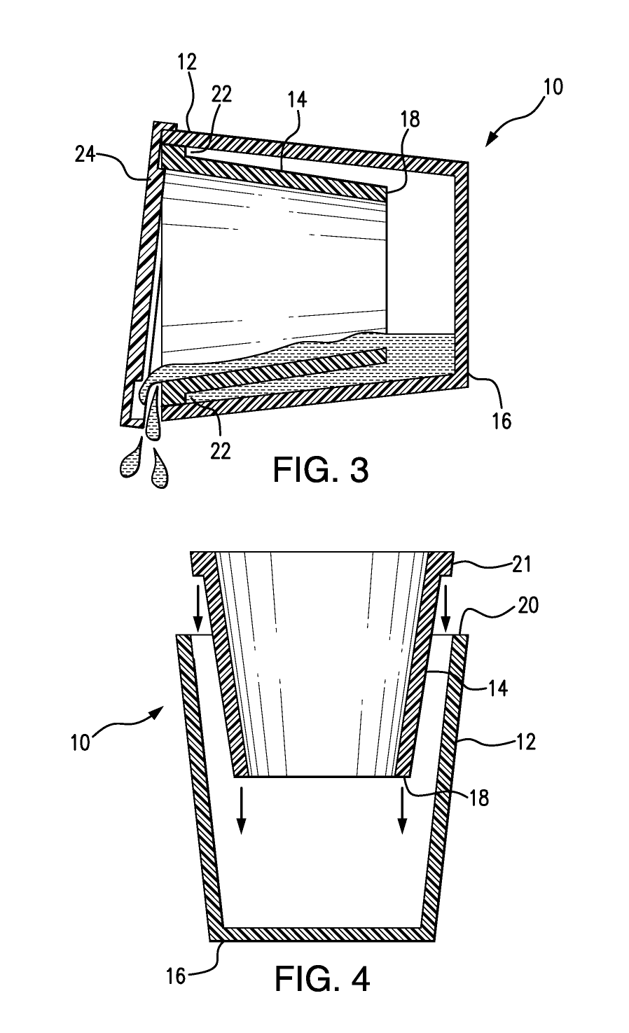

Referring to FIG. 3, in the event that the snap-fit lid 24 of the urine specimen collection device 10 is not adequately secured to the container 12 (as shown in FIG. 3) when in transport, at least a portion of the specimen will be trapped in the pocket 22 and not exit the container 12 via an opening between the upper rim 20 and the lid 24. The pocket 22 surrounding the inner shell 14 is particularly sized and configured to capture a volume of the urine specimen suitable for testing. For manufacturing purposes, the inner shell 14 may be adapted for use in combination with urine sample containers that are presently known in the art, wherein the inner shell 14 is fixed to the urine sample container as described above. While not shown, a further embodiment is fully contemplated within the spirit and scope of the invention wherein a point of care chemical reagent test indicator is fitted between the inner shell 14 and the outer wall of the container 12 for rapid detection of the presence of one or more substances in the collected urine. The point of care chemical reagent test indicator can be fitted within the container during the manufacturing process.

Referring to FIG. 4, the urine sample collection device 10 is manufactured by fixing the top end 21 of the inner shell 14 to the upper rim 20 of the container 12. The inner shell 14 may be fixed to the upper rim 20 using any suitable method, such as heat seal or use of an adhesive or bonding agent. In one embodiment, the container 12 and inner shell 14 are tapered, wherein the inner shell 14 is tapered slightly more so than the container 12, such that the top end 21 of the inner shell 14 is wedged against the upper rim 20 of the container 12 to produce a tighter seal, which may be supplemented using the methods described above (e.g., heat seal, adhesive, and bonding agent).

As shown throughout the Figures, pouring of a urine sample via the open end of the container 12 is permitted where a standard volume of the urine sample exists. Moreover, the center of the container 12 is not obstructed and permits use of a pipette to extract a urine sample therefrom.

While the present invention has been shown and described in accordance with several preferred and practical embodiments thereof, it is recognized that departures from the instant disclosure are fully contemplated within the spirit and scope of the invention as defined in the following claims and as interpreted under the Doctrine of Equivalence.

What is claimed is:

1. A urine sample collection device comprising:
a container surrounding an inner cavity, and said container having a closed bottom end with one or more wall surfaces extending upwardly therefrom and terminating at an upper rim surrounding an open top end;
an inner shell surrounding an inner channel extending between opposing open ends of said inner shell, and said inner shell being fixable to said container such that said inner shell extends from the upper rim into the cavity of the container, and said inner shell being sized and configured to form a pocket between said inner shell and the one or more wall surfaces of said container;
a lid being releasably securable to said container for covering the open top end; and
wherein a portion of a volume of fluid in said container will become trapped in said pocket between said inner shell and the one or more wall surfaces of said container when there is an opening between said lid and the upper rim of said container permitting exit of the volume of fluid therethrough.

2. The urine sample collection device as recited in claim 1 wherein said pocket is sized and configured for trapping a sufficient volume of fluid for testing.

3. The urine sample collection device as recited in claim 1 wherein said inner shell is tapered in relation to the one or more wall surfaces of said container.

4. The urine sample collection device as recited in claim 1 wherein said inner shell is convexly curved in relation to the one or more wall surfaces of said container.

5. The lid as recited in claim 1 wherein said lid is a snap-fit lid.

6. The urine sample collection device as recited in claim 1 wherein said inner shell is fixed to the upper rim of said container by heat seal.

7. The urine sample collection device as recited in claim 1 wherein said inner shell is fixed to the upper rim of said container by an adhesive.

8. The urine sample collection device as recited in claim 1 wherein said inner shell is fixed to the upper rim of said container by a bonding agent.

9. A urine sample collection device comprising:
a container surrounding an inner cavity, and said container having a closed bottom end with one or more wall surfaces extending upwardly therefrom and terminating at an upper rim surrounding an open top end;
an inner shell surrounding an inner channel extending between opposing open ends of said inner shell, and said inner shell being fixable to said container such that said inner shell extends from the upper rim into the cavity of the container, and said inner shell being sized and configured to form a pocket between said inner shell and the one or more wall surfaces of said container; and
wherein a portion of a volume of fluid in said container will become trapped in said pocket between said inner shell and the one or more wall surfaces of said container and not exit the cavity of the container when the closed bottom surface of said container is not parallel with the ground surface.

10. The urine sample collection device as recited in claim 9 wherein said pocket is sized and configured for trapping a sufficient volume of fluid for testing.

11. The urine sample collection device as recited in claim 9 wherein said inner shell is tapered in relation to the one or more wall surfaces of said container.

12. The urine sample collection device as recited in claim 9 wherein said inner shell is convexly curved in relation to the one or more wall surfaces of said container.

13. The urine sample collection device as recited in claim 9 further comprising a lid being releasably securable to said container for covering the open top end.

14. The urine sample collection device as recited in claim 13 wherein said lid is a snap-fit lid.

15. The urine sample collection device as recited in claim 9 wherein said inner shell is fixed to the upper rim of said container by heat seal.

16. The urine sample collection device as recited in claim 9 wherein said inner shell is fixed to the upper rim of said container by an adhesive.

17. The urine sample collection device as recited in claim 9 wherein said inner shell is fixed to the upper rim of said container by a bonding agent.
